(19) United States
(12) Patent Application Publication
(10) Pub. No.: US 2006/0015082 A1
(43) Pub. Date: Jan. 19, 2006

(54) Male urinary incontinence apparatus

(76) Inventors: Jack Pearson, Ft. Lauderdale, FL (US); Jody Pearson, Fr. Lauderdale, FL (US)

Correspondence Address: Mark D. Bowen;Stearns Weaver Miller, et al., Suite 1900, 200 East Broward Boulevard, Fort Lauderdale, FL, 33301, US

(21) Appl. No.: 10/891,741

(22) Filed: Jul. 15, 2004

(51) Int. Cl. A61F 5/44

(52) U.S. Cl. 604/347

(57) ABSTRACT

An apparatus for use in covering relation with the male penis to manage incontinence is disclosed. The apparatus includes a generally tubular sheath adapted for use in covering relation over the user's penis. The sheath includes a tapered end having a tip adapted for connection to a tube to facilitate fluid communication between the sheath interior and a urine collection bag that is preferably secured to the lower portion of the user's leg. The sheath is preferably fabricated from a soft, rubber-like material in a double wall configuration wherein inner and outer layers combine to form an interstitial space. The interstitial space may be filled with a liquid or gel substance for increased comfort. The device is further adapted with projecting flaps and is adapted to be secured by a suitable fastening system, such as a strap or other securing means connected to the flaps.

### CROSS REFERENCE TO RELATED APPLICATIONS

N/A

### STATEMENT REGARDING FEDERALLY SPONSORED RESEARCH OR DEVELOPMENT

N/A

### COPYRIGHT NOTICE

A portion of the disclosure of this patent document contains material that is subject to copyright protection. The copyright owner has no objection to the facsimile reproduction by anyone of the patent document or patent disclosure, as it appears in the Patent and Trademark Office patent file or records, but otherwise reserves all copyrights.

### BACKGROUND OF THE INVENTION

1. Field of the Invention

The present invention relates to incontinence, and more particularly, to an apparatus for use by males suffering from incontinence problems.

2. Description of the Background Art

Incontinence is a growing problem, particularly in adult males ages 60 and older. Approximately two million males in the United States suffer moderate to severe incontinence. Common causes for this condition include an enlarged prostate, prostate cancer, pelvic trauma, spinal cord injury, and medication side effects. Over $15 billion is spent annually on incontinence related care.

There are many male urinary incontinence management devices on the market ranging from the intrusive urinary tract catheters, to diapers. Neither of these devices, however, offer safe, comfortable, and non-traumatic control or management of male urinary incontinence. The use of urinary tract catheters is often problematic due to their intrusive nature and resulting occurrence of urinary tract infections. On the other hand, the use of diaper-type devices often causes skin irritation and rashes. In addition, diaper-type devices are not effective in maintaining an accurate measure of a patient's fluid intake and output.

The present invention addresses the limitations of certain prior art devices used for managing male incontinence. Specifically, the invention provides a wearable external device that is both effective and easy to use, and which will remain generally unnoticed underneath the clothing of the wearer without creating a feeling of self-consciousness. While not a cure for male urinary incontinence, the invention is intended to allow for a better quality of life with less skin irritation and fewer problems as compared to present, commercially available and accepted incontinent devices.

### BRIEF SUMMARY OF THE INVENTION

The present invention addresses and overcomes the limitations and disadvantages in the art by providing an apparatus for use in covering relation with the male penis to manage incontinence. The apparatus includes a generally tubular sheath adapted to be worn in covering relation over the user's penis. The sheath includes a tapered end or tip adapted for connection to a tube to facilitate fluid communication between the sheath interior and a urine collection bag that is preferably secured to the lower portion of the user's leg, or other drainage system such as a Foley catheter drainage bag. The sheath is preferably fabricated from a soft, rubber-like material, and preferably in a double wall configuration wherein inner and outer layers combine to form an interstitial space. The interstitial space may be filled with a liquid or gel substance for increased comfort. In an alternate embodiment, however, the sheath may be fabricated from a single layer of suitable material, such as silicone of latex. A significant aspect of the present invention relates to securing the sheath by a suitable fastening system. More particularly, a waist-strap or belt-like structure is disclosed as a securing means. The waist-strap securing means eliminates the risk of dislodgement of the sheath from the penis (hence incontinence).

Accordingly, it is an object of the present invention to provide an improved apparatus for controlling male incontinence.

Another object of the present invention is to provide an apparatus for controlling male incontinence that is comfortable and easy to use.

Still these and other objects will become apparent with reference to the detailed description below and the accompanying drawings.

### BRIEF DESCRIPTION OF THE SEVERAL VIEWS OF THE DRAWINGS

FIG. 1 is front perspective view of an incontinence apparatus and strap according to the present invention;

FIG. 2 is a rear perspective view thereof;

FIG. 3 is rear perspective view thereof without the strap;

FIG. 4 is a side view thereof;

FIG. 5 is a perspective view of an alternate embodiment;

FIG. 6 is a partially sectioned view thereof;

FIG. 7 is a side perspective view of an alternate embodiment incontinence apparatus having an alternate securing means;

FIG. 8 is a rear perspective view thereof;

FIG. 9 is a side perspective view of yet another alternate embodiment; and

FIG. 10 is perspective view of yet another embodiment.

### DETAILED DESCRIPTION OF THE INVENTION

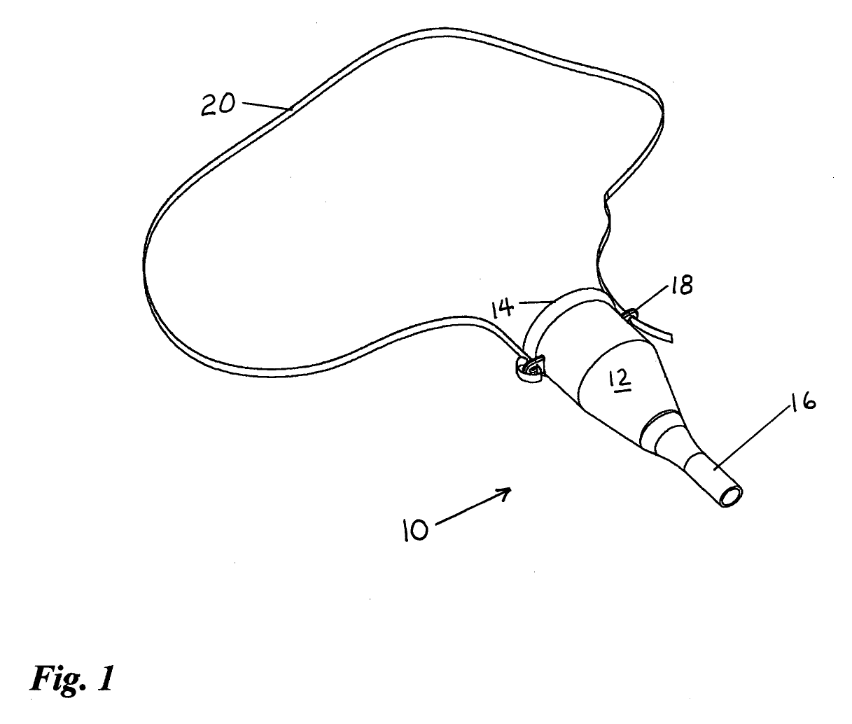

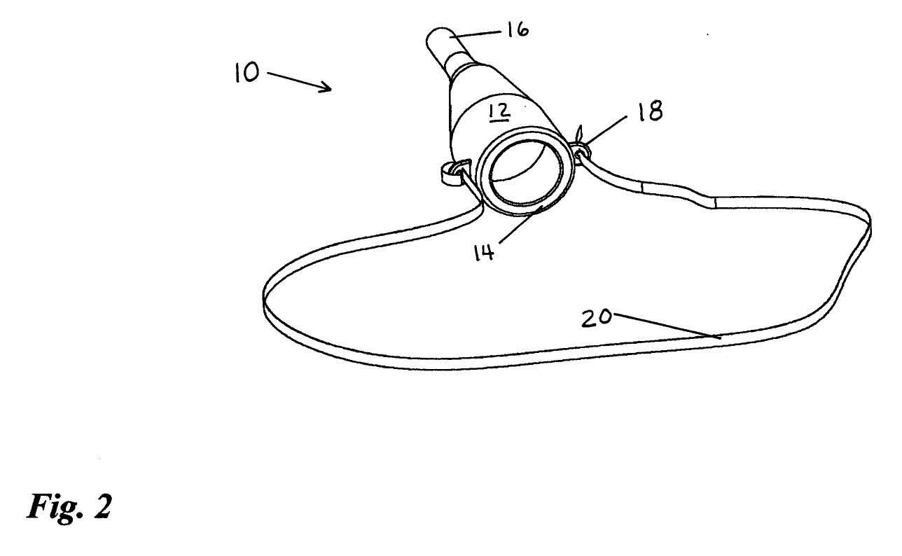

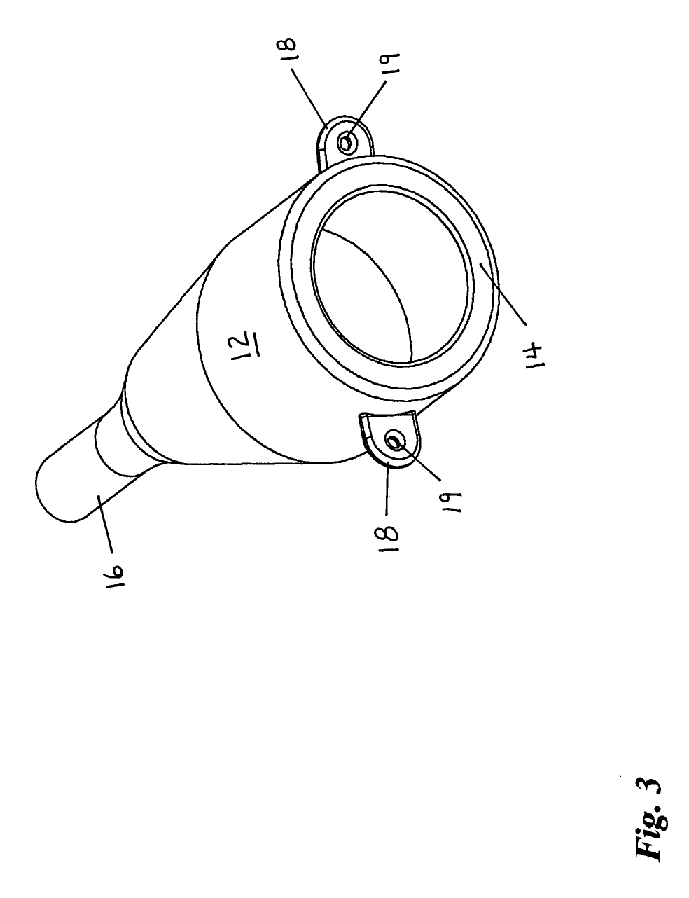

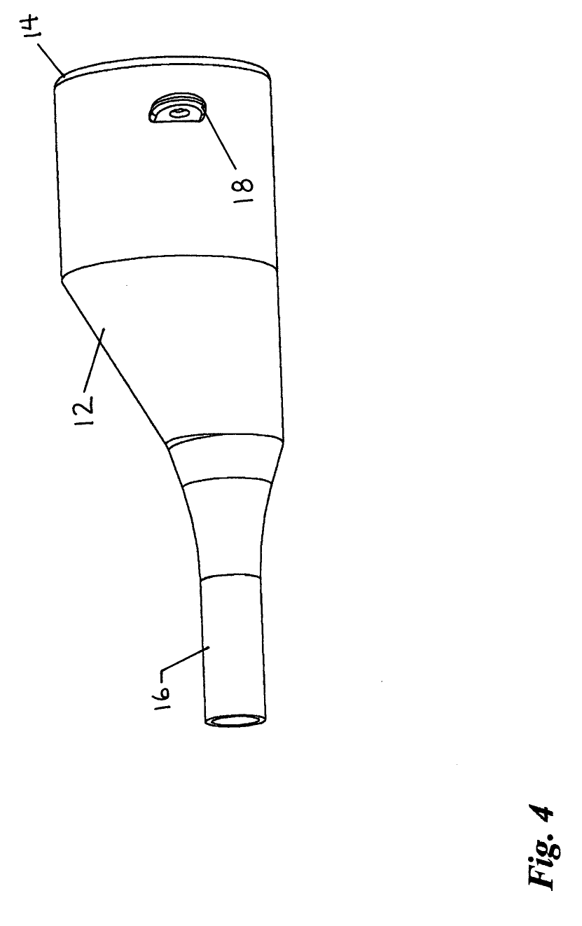

With reference now to the drawings FIGS. 1-4 depict a preferred embodiment of an incontinence management apparatus, generally referenced as 10, for use in covering relation with the male penis to manage incontinence. Incontinence management apparatus 10 includes a generally tubular sheath 12 adapted for fitting in covering relation over the user's penis (not shown). Sheath 12 preferably includes an first/proximal end 14 defining an opening, and a second/distal end 16 defining a tapered tip adapted for connection to a tube (not shown) to facilitate fluid communication between the sheath interior and a urine collection bag, such as a Foley catheter drainage bag or drainage system (not shown) secured to the lower portion of the user's leg as is customary in the art of incontinence management. Sheath 12 is preferably fabricated from a soft, rubber-like material, such as silicone or latex, however, any suitable material is considered within the scope of the invention. In an alternate embodiment, sheath first proximal end portion 14 may comprise a fabric body and the second distal end 16 may comprise a soft rubber-like material. In other embodiments, sheath 12 may be fabricated from a soft, rubber-like material wherein the proximal end includes a fabric liner. Sheath 12 further includes a pair of projecting flaps, referenced as 18, each of which define an aperture 19. Projecting flaps 18 function as mounting structures for connection of a strap 20. The use of projecting flaps is considered significant as the flaps place the strap connection points in spaced relation from the user's penis and further allow for movement of the straps to conform to the user's movement thereby maximizing comfort. Strap 20 is preferably elastic or soft fabric such as spandex, and secures sheath 12 to the user's body, in covering relation with the user's penis, by wrapping around the user's waist.

As discussed above, sheath 12 is preferably fabricated from a soft, FDA approved silicon material, in a double walled tubular configuration adapted to be filled with a gel, water, or any other suitable substance. More particularly, sheath 12 is preferably fabricated as an open ended, generally tubular structure, having radially inner and outer sleeve members sealingly connected to form a double wall sheath defining an interior volume for receiving the user's penis. The inner and outer sleeve members are preferably sealed along common peripheral edges by heat sealing and/or ultrasonic welding. Once formed the double wall structure defines an internally enclosed interstitial space that may be filled with a liquid or gel substance for increased comfort. The combination of soft and pliable inner and outer sleeve members containing a liquid or gel substance results in an apparatus that may be comfortably worn over the user's penis.

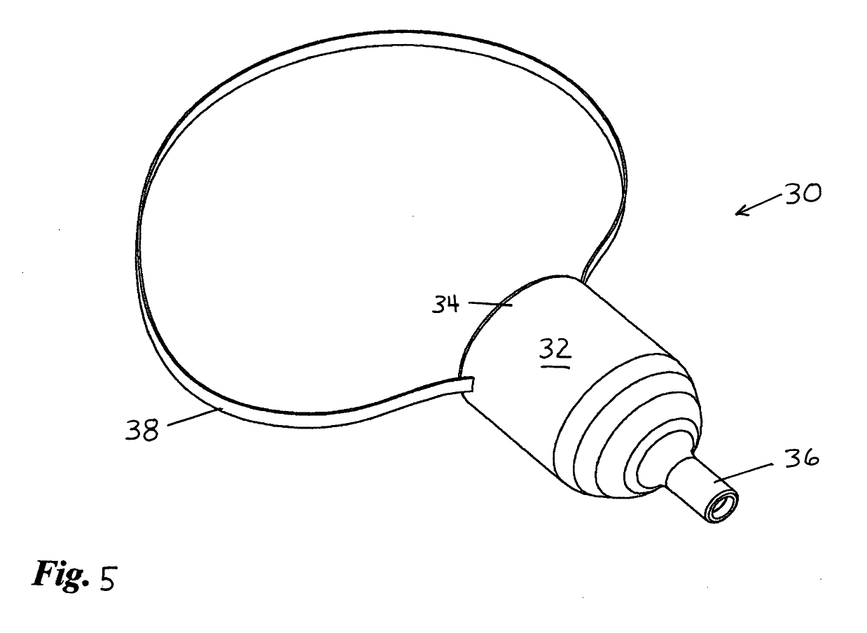

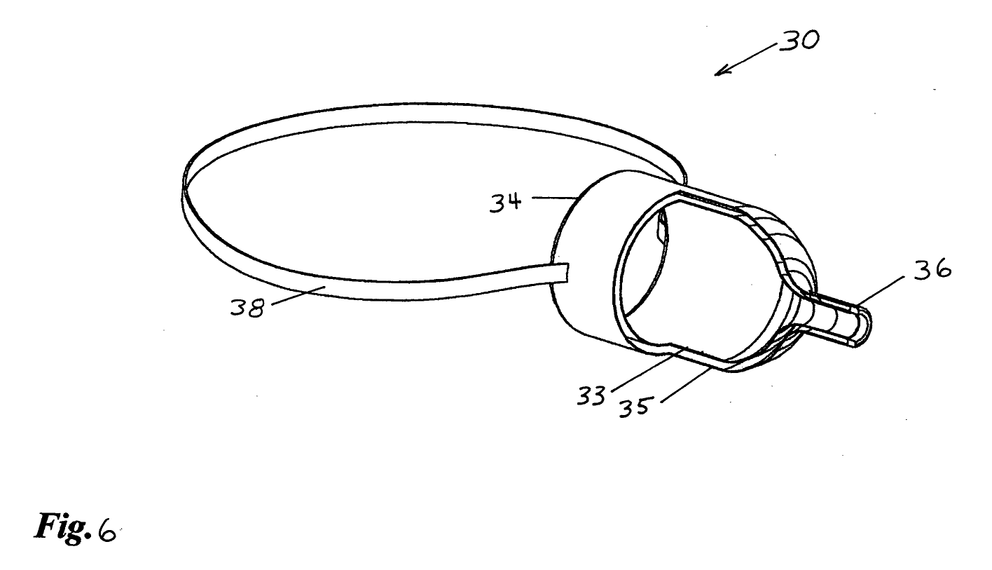

FIGS. 5 and 6 depict an alternate embodiment male incontinence apparatus, referenced as 30. Incontinence management apparatus 30 comprises a generally tubular sheath 32 adapted for fitting in covering relation over at least a portion of the user's penis (not shown). Sheath 32 preferably includes an first/proximal end 34 defining an opening, and a second/distal end 36 defining a tapered tip adapted for connection to a tube (not shown) to facilitate fluid communication between the sheath interior and a urine collection bag (not shown) that is preferably secured to the lower portion of the user's leg as is customary in the art of incontinence management. Sheath 32 further includes a strap 38. Strap 38 is preferably elastic or soft fabric, and secures sheath 32 to the user's body, in covering relation with at least a portion of the user's penis, by wrapping around the user's waist. In alternate embodiments, strap 38 may comprise a soft fabric having a hook and loop fastening mechanism (e.g. Velcro®).

Sheath 32 is preferably fabricated from a soft, FDA approved silicone material, in a double walled tubular configuration adapted to be filled with a gel, water, or any other suitable substance. As noted herein above, the sheath may also incorporate fabric sections, however, the FDA approved silicone, or other rubberized material, is considered preferable for use at the distal end or tip. As best depicted in sectional view of FIG. 6, sheath 32 is preferably fabricated as an open ended, generally tubular structure, having an inner sleeve 33 and an outer sleeve 35 radially spaced from inner sleeve 33 to form an interstitial space. The inner and outer sleeves are preferably sealed along common peripheral edges by heat sealing and/or ultrasonic welding. Once formed the double wall structure defines an internally enclosed interstitial space that may be filled with a liquid or gel substance for increased comfort. The combination of soft and pliable inner and outer sleeve members containing a liquid or gel substance results in an apparatus that may be comfortably worn over the user's penis.

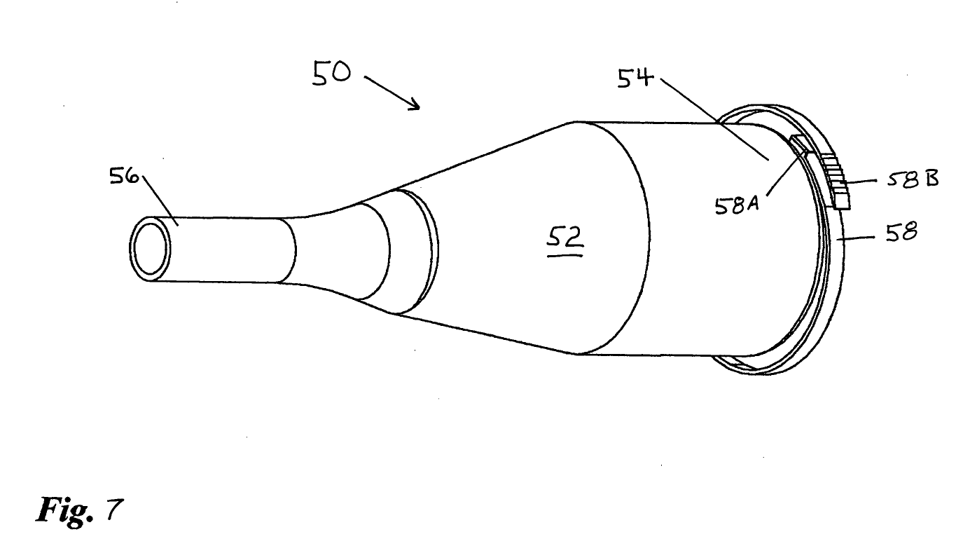

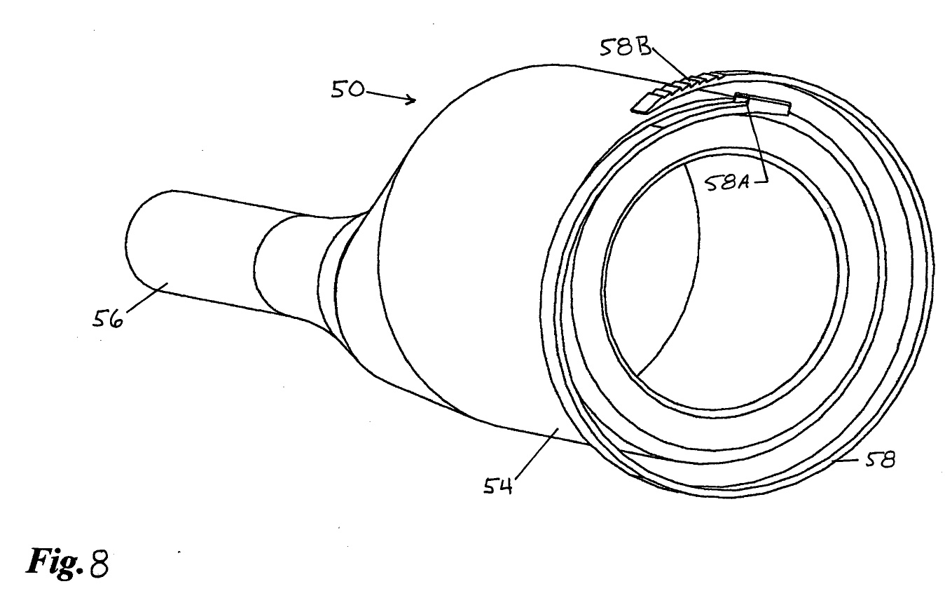

FIGS. 7-8 depict a male incontinence apparatus, referenced as 50, having an alternate securing mechanism. More particularly, male incontinence apparatus 50 comprises a generally tubular sheath 52 adapted for fitting in covering relation over at least a portion of the user's penis (not shown). Similar to the embodiments previously disclosed herein, tubular sheath 52 includes an first/proximal end 54 defining an opening, and a second/distal end 56 defining a tapered tip adapted for connection to a tube (not shown) to facilitate fluid communication between the sheath interior and a urine collection bag (not shown) that is preferably secured to the lower portion of the user's leg, or Foley drainage bag, as is customary in the art of incontinence management. Sheath 52 further includes a circumferential strap 58 disposed at or in proximity to the first/proximal end 54 of sheath 52. Circumferential strap 58 preferably includes a first end portion 58A adapted with a locking member, and a second end portion 58B adapted with projecting teeth. Circumferential strap 58 functions to secure apparatus 50 to the user by causing radially inward deformation of proximal end 54 upon tightened, releasable locking engagement of strap ends 58A and 58B.

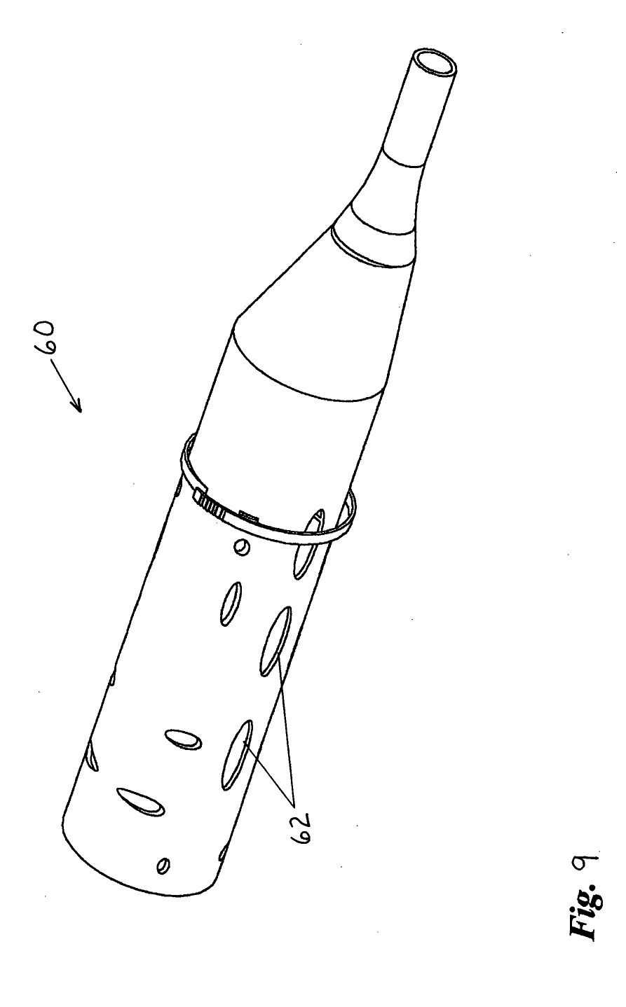

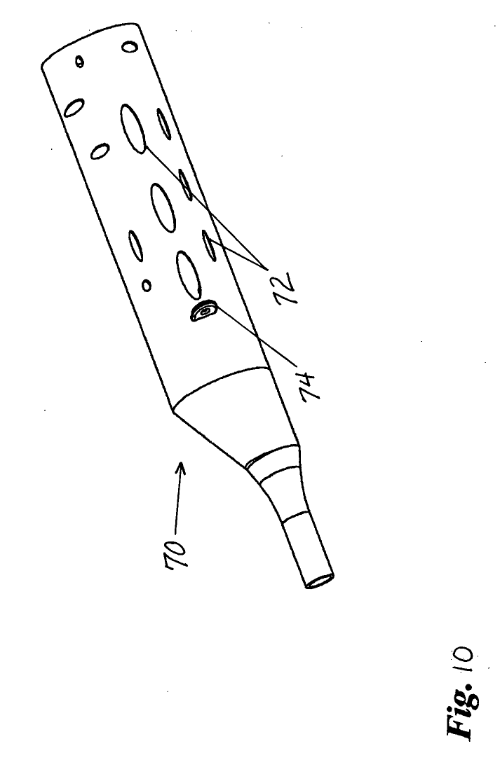

Finally, FIGS. 9 and 10 depict additional embodiments for an apparatus for managing incontinence in males. FIG. 9 depicts such an apparatus, referenced as 60, wherein the apparatus defines a plurality of apertures 62 in the double wall structure which function to provide ventilation for increased comfort. Apparatus 60 further includes a circumferential strap 64 for securing apparatus 60 to the user when in use. As best illustrated by FIG. 9, circumferential strap 64 may be slidably disposed along the length of the apparatus to allow for user positioning thereof, or may be fixed at any suitable location. FIG. 10 depicts an embodiment of the apparatus, referenced as 70. Apparatus 70 is similar in structure to the apparatus 60 depicted in FIG. 9, to the extent that apparatus 70 is depicted with apertures 72. However, apparatus 70 is adapted with opposing radially projecting flaps 74 and is thus adapted for use with a strap to secure apparatus 70 to the user. Thus, a primary advantage of the present invention is to provide a male incontinence apparatus that is secured to the user by a waist-strap or belt that prevents the apparatus from being dislodged from the user's penis thereby effectively eliminating the risk of incontinence.

As should now be apparent, each of the various embodiments disclosed herein are suitable for use in the management of incontinence in males by placement of one of said apparatus over the male penis so as to be in substantial covering relation therewith and securing the apparatus using the provided securing structure. Once the apparatus is fitted on and secured to the user, the apparatus may be connected to a urine collection bag affixed to the user's leg by plastic tubing connected to the apparatus distal end such that discharged urine flows out of the distal end of the apparatus through the tubing and into collection bag.

The instant invention has been shown and described herein in what is considered to be the most practical and preferred embodiment. It is recognized, however, that departures may be made therefrom within the scope of the invention and that obvious structural and/or functional modifications will occur to a person skilled in the art.

What I claim is:

1. A male urinary incontinence management apparatus comprising:
a generally tubular sheath having a proximal end defining a first opening and a distal end defining a second opening;
said sheath including radially spaced inner and outer walls defining an interstitial space;
a substance substantially filling said interstitial space;
said tubular sheath including opposing radially projecting flaps;
means, connected to said opposing radially projecting flaps, for securing said sheath in covering relation with the male penis.

2. A male urinary incontinence management apparatus according to claim 1, wherein said substance comprises a liquid.

3. A male urinary incontinence management apparatus according to claim 1, wherein said substance comprises a gel.

4. A male urinary incontinence management apparatus according to claim 1, wherein said inner and outer walls are formed of an FDA approved material.

5. A male urinary incontinence management apparatus according to claim 4, wherein said FDA approved material comprises silicone.

6. A male urinary incontinence management apparatus according to claim 1, wherein said means for securing said sheath includes an elastic strap having opposing ends connected to said sheath.

7. A male urinary incontinence management apparatus according to claim 1, wherein said means for securing said sheath includes a circumferential strap connected to said sheath and disposed between said first and second ends.

8. A male urinary incontinence management apparatus comprising:
a generally tubular sheath having a proximal end defining a first opening, a distal end defining a second opening, and an interior volume communicating with said first and second openings;
said sheath including radially spaced inner and outer concentrically disposed walls defining an interstitial space between said inner and outer walls;
said inner and outer walls defining a plurality of through bores for providing ventilation;
a substance substantially filling said interstitial space between said inner and outer walls;
a strap for securing said sheath in covering relation with the male penis.

9. A male urinary incontinence management apparatus according to claim 8, wherein said substance comprises a liquid.

10. A male urinary incontinence management apparatus according to claim 8, wherein said substance comprises a gel.

11. A male urinary incontinence management apparatus according to claim 8, wherein said inner and outer walls are formed of an FDA approved material.

12. A male urinary incontinence management apparatus according to claim 11, wherein said FDA approved material comprises silicone.
